(19) United States
(12) Patent Application Publication
(10) Pub. No.: US 2008/0154132 A1
(43) Pub. Date: Jun. 26, 2008

(54) Method and Apparatus for the Visualization of the Focus Generated Using Focused Ultrasound

(75) Inventors: Christopher Hall, Hopewell Junction, NY (US); Shunmugavelu Sokka, New Rochelle, NY (US); David L.M. Savery, Tarrytown, NY (US); Chien T. Chin, Tarrytown, NY (US); Michalakis Averkiou, Kirkland, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/816,490

(22) PCT Filed: Feb. 6, 2006
(86) PCT No.: PCT/IB06/50382

§ 371 Date: Aug. 16, 2007

Related U.S. Application Data
(60) Provisional application No. 60/653,873, filed on Feb. 17, 2005.

(51) Int. Cl. A61B 8/00

(52) U.S. Cl. 600/439

(57) ABSTRACT

The present invention relates to monitoring the focus of a therapeutic transducer in an interactive real-time manner by turning off the ultrasound transmits of the imaging transducer but continuing to receive in all directions with an imaging probe in order to identify the therapeutic beam focus. The therapeutic focused beam acts as the only transmit and as long as there is a scatterer at the focus, a strong receive signal will be generated to identify the focus.

The present invention relates to a method and an apparatus for monitoring the position of the focus of a therapeutic ultrasound transducer in an interactive real-time manner to improve treatment times and increase the accuracy of focused ultrasound procedures. In particular, the present invention relates to monitoring the focus of a therapeutic transducer in an interactive real-time manner by turning off the ultrasound transmits of the imaging transducer but continue to receive in all directions with an imaging probe to identify the therapeutic beam focus. The therapeutic focused beam acts as the only transmit and as long as there is a scatterer at the focus, a strong receive signal will be generated to identify the focus.

Ablation can be divided into two basic categories: chemical and thermal. In chemical ablation, tissue toxic agents, such as absolute alcohol or acetic acid, are injected directly into the tissues to be ablated. And in thermal ablation, dysfunctional tissue is destroyed by thermal means via energy delivered by radio frequency electromagnetic waves, microwaves, ultrasound, laser or hot liquids. All of these energy delivery mechanisms rely on the tissue to absorb the energy in the form of heat until proteins denature and cell death results. In addition to these heating methods, thermal ablation includes cryotherapy, which destroys tissue by freezing.

These chemical and thermal ablation techniques have been applied in almost every major organ system to treat benign and malignant disease: liver, heart, prostate, kidneys, lungs, and the brain among the most prominent. In order for ablation to be minimally or non-invasive, the ablation energy must be delivered with minimal intervention and minimal damage to surrounding tissue. Chemical ablation, cryotherapy, laser, RF, and microwave ablation are typically done via percutaneous needles or intravascular catheters. The treatment needle containing the active element is inserted into the tumor through the skin on the treatment catheter is directed through the vasculature to the target location. With ultrasound and in some cases microwave, the energy can be directed towards or focused at the location without direct contact and can, therefore, be non-invasive. Regardless of the energy delivery mechanism, a key component of ablation therapy is imaging.

Imaging systems have been critical to the acceptance of minimally invasive ablation technologies. Imaging is utilized in every step of the ablation process. First, imaging is used in treatment planning. In this phase, the target tissue is identified and the physical approaches, to the tumor, avoiding critical structures, are identified. Second, imaging is used to guide the placement of the ablation device relative to the target tissue, whether it is the needle, the catheter, or an external device. Next, imaging can be used to monitor the therapy to track progress and to provide feedback to make energy level and dose adjustments. Finally, imaging is used after ablation to assess the resulting lesion size and lesion boundaries, which are important metrics on the effectiveness of the ablation treatment. Of these four major thrusts of imaging in ablation therapy, treatment planning and image guided placement of the ablation device are the most mature techniques in terms of both research and clinical practice and have made minimally invasive ablation procedures possible. However, the future of ablation depends on the development of imaging techniques to monitor therapy and provide immediate feedback. Such monitoring techniques will have a dramatic impact on procedure cost and successful clinical outcomes.

Almost every imaging modality has been investigated for ablation imaging. For years, standard X-ray has been used for treatment planning, and X-ray fluoroscopy has been used to guide RF ablation catheters for cardiac applications. More recently, MRI, X-ray CT, and ultrasound have also been used for treatment planning and needle or catheter guidance. Recent advances in MR imaging have made accurate spatial temperature imaging possible, and hence thermal ablation monitoring and feedback with MRI is now being utilized for ultrasound ablation and cryotherapy. However, probably the most promising imaging modality for monitoring of ablation is ultrasound. Ultrasound is significantly less expensive than MRI, does not employ ionizing radiation like X-ray based imaging modalities, is already used to guide several ablation therapies, and is real-time; all making ultrasound imaging ideally suited for the monitoring and post ablation assessment phases of ablation imaging.

Focused ultrasound involves the use of highly focused sound waves to cause localized low temperature heating (hyperthermia) of tissue or possible ablation/destruction of tissue (high intensity focused ultrasound—HIFU). Focused ultrasound (FUS) is currently being examined as an alternative means for treating patients with a wide-variety of illnesses including cancerous growth and heart conduction pathologies. FUS is currently employed in China for treating over 1,000 patients with very promising outcomes [1]; is under trial in England [2]; and has just completed Phase III trials in the United States for uses in treating benign prostate hyperplasia and in the treatment of uterine fibroids [3].

One of the challenges with the remote interaction of focused ultrasound and tissue is monitoring of the location of the therapeutic delivery of sound before an actual dose is delivered.

Previously, several techniques have been tried which involve the use of clinical imaging system such as MRI and ultrasound. One technique uses MRI monitoring of temperature changes in tissue from the actual delivery of therapeutic sound [4, 5]. Although providing useful information, the difficulty for use in an intra-operative environment and the expense of using an MRI clinical scanning system and MR compatible tools discourages this approach. A second technique utilizes ultrasound as a monitoring tool but calculates the position of the focus of the therapeutic device from assumptions about the acoustic properties of tissue such as the speed of sound propagation. This approach can be valuable for obtaining a rough idea of the location of the focus, but does not allow for organ/tissue variability in acoustic properties, which can be critical when temperature gradients exist.

Current techniques for the visualization of focused ultrasound rely on MRI based techniques that provide slow interaction for the positioning of the focus. The current invention would allow for an intuitive, interactive monitoring and guidance of the position of therapeutic delivery prior to actual delivery of the dose.

Diagnostic ultrasound as a monitoring and guidance tool provides one of the most inexpensive imaging modalities. Current proposed solutions include the use of MRI that can be resources (time, people, and hospital floor space) intensive.

It would be desirable to provide a focused ultrasound system that avoids the aforementioned drawbacks of the prior art proposals while providing an inexpensive real-time diagnostic imaging system capable of being operated in both traditional imaging mode as well as a passive receiver.

It is therefore desirable to provide a method and an apparatus for monitoring the position of the focus of a therapeutic transducer in an interactive, real-time manner that will improve traditional treatment times and increase the accuracy of focused ultrasound procedures.

It is desirable to provide a method and an apparatus for monitoring the focus of a therapeutic transducer in an interactive, real-time manner by turning off the ultrasound transmits of the imaging transducer but continuing to receive in all directions with an imaging probe in order to identify the therapeutic beam focus. The therapeutic focused beam acts as the only transmit and as long as there is a scattering phenomenon at the focus, a strong receive signal will be generated to identify the focus.

Other objects of the invention will become clear and apparent from the foregoing description and accompanying drawings.

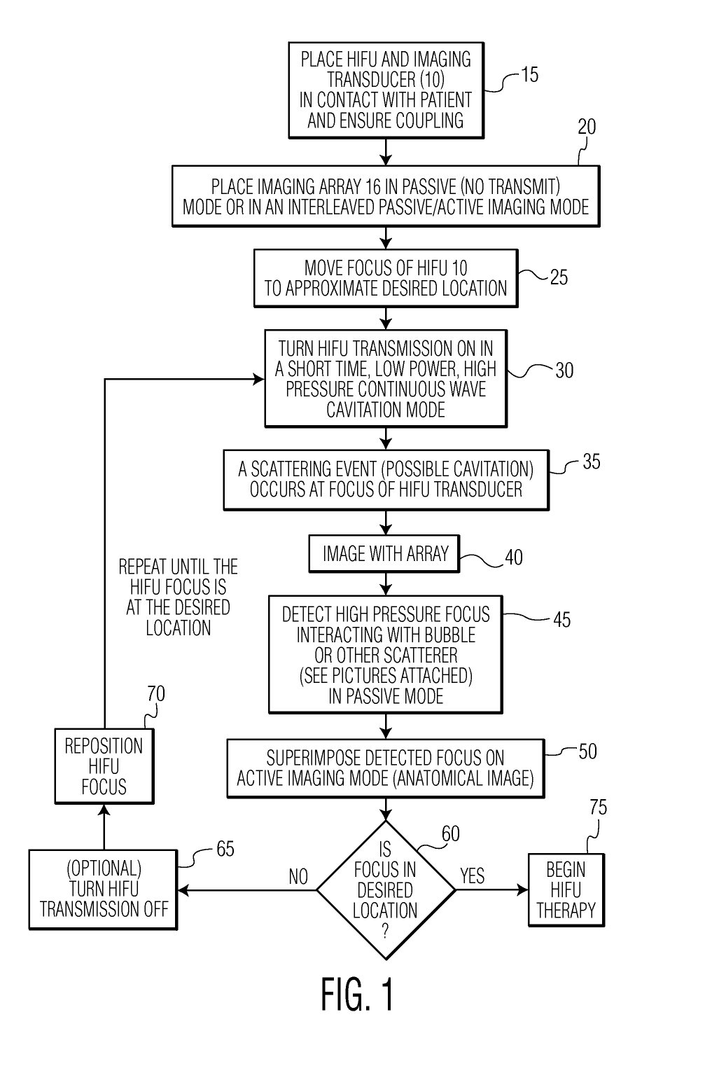

FIG. 1 is detailed flow chart describing operation of the present invention:

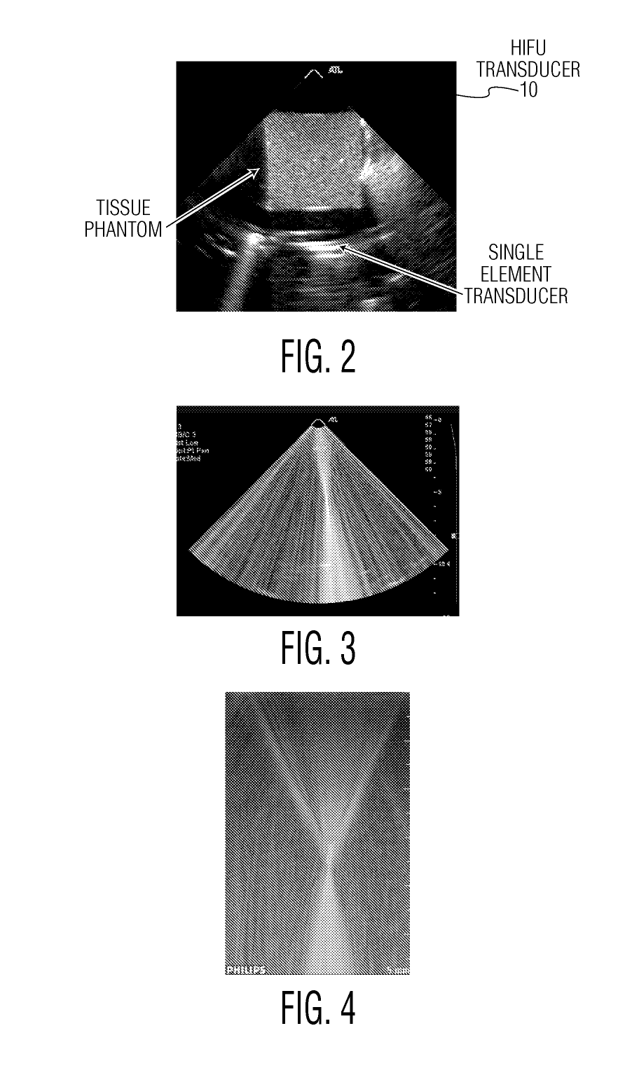

FIG. 2 illustrates the transducer of the present invention;

FIG. 3 illustrates focus visualization with non-zero transmit on a sector phased array in accordance with the teachings of the present invention; and

FIG. 4 illustrates focus visualization with transmit=0.0 on a linear array in accordance with the teachings of the present invention.

Before explaining the disclosed embodiments of the present invention in detail, it is to be understood that the invention is not limited in its application to the details of the particular arrangements shown since the invention is capable of other embodiments. Also, the terminology used herein is for the purpose of description and not of limitation. Referring now to the drawings of FIGS. 1-4, FIG. 1 provides a detailed flow chart showing the operation of the present invention for a focused ultrasound transducer 10 (see FIG. 2) that is coupled to a patient's body by means of an ultrasound coupling medium such as gel and/or degassed water.

The transducer is operated in a high power mode that is sufficient for it to interact with tissue and cause temporary but reversible changes in the tissue in order to guide the placement of the focus of the transducer. These changes cause localized scatter of sound in the specific location of the focus of the transducer. This increased ultrasonic scatter may be due to the interaction of the high intensity ultrasound with scatters at the transducer's focus, induced formation of the microscopic and macroscopic bubbles, or due to the changes in tissue due to the local temperature change. The formation of the bubbles has been noted the article “Real time Visualization of High-Intensity Focused Ultrasound treatment Using Ultrasound Imaging by S. Vaezy, X. Shi, R. Martin, E. Chi, P. Nelson, M. Bailey and L. Crum, Ultrasound In Med. & Bio., Vol. 27, No. 1, 33-42 2001, which noted that:

“It is highly likely that the bright hyperechoic spot at the HIFU focus is due to gas and/or vapor bubbles. The low acoustic impedance of bubbles (several orders of magnitude less than tissue) makes them appear hyperechoic and, thus a good candidate responsible for the observed hyperechoic region at the focal spot. In fact, during the HIFU treatment, we have observed the escape of bright speckle-size spots (suspected to be bubbles) from the focal spot into the vascular system of the liver.” (p. 40)

While this article notes this scattering effect due to the bubbles, its does not seek to utilize or provide a mechanism for utilizing this effect to improve monitoring the position of the focus of a therapeutic transducer in an interactive real-time manner to improve treatment times and increase the accuracy of focused ultrasound procedures as provided by the present invention.

Referring to FIG. 1, as indicated in step 15, the transducer 10 is a High Intensity Focused Ultrasound (HIFU) and Imaging Transducer 10 that is placed in contact with a patient and coupling is ensured, as mentioned previously, by use of an ultrasonic coupling medium such as gel and/or degassed water. An imaging array 16 is placed in either a passive (no transmit mode) or an interleaved passive/active imaging mode as indicated in step 20 of FIG. 1.

The focus of the HIFU transducer 10 is moved to an approximate desired location, namely so that the focus will be positioned as approximated in the tissue of intent (Step 25 of FIG. 1).

The HIFU transducer 10 is turned on for a short time, low power, high pressure, continuous wave, and may or may not induce cavitation (Step 30 of FIG. 1). The short time interval can vary from microseconds to tens of seconds. The low power—acoustic power for transducer 10—can vary from milliwatts to 10 watts. The high pressures can vary from 100s of kiloPascals to the 10s of MegaPascals.

A scattering event or phenomenon (possibly cavitation) will occur at the focus of the HIFU transducer (Step 35 of FIG. 1). As stated previously, the increased ultrasonic scatter may be due to the interaction of the high intensity ultrasound with scatters at the transducer's focus, induced formation of microscopic and macroscopic bubbles (cavitation) or due to the change in tissue due to local temperature increase. The scattering may be due to calcification, interface layer of skin and fat, of muscle and fat, of muscle and tendon, or local tissue phenomenon such as debris at the tissue, a tumor, or any tissue anomaly.

The image array 16 will image in its passive mode or interleaved mode (Step 40 of FIG. 1), receive beam formed, and will detect (Step 45 of FIG. 1) the high pressure focus or other scatterer. The detected focus will be superimposed on the active imaging mode (anatomical image) of the imaging array screen (Step 50 of FIG. 1). In this manner, it can be determined if the focus is in the correct location (Step 60 of FIG. 1) and if the HIFU therapy (Step 75 of FIG. 1) can begin. If not, then the transducer's transmission may be turned off (Step 65 of FIG. 1), the HIFU transducer 10 can be moved to reposition the focus to the correct location based on the detected location by the imaging array (Step 70 of FIG. 1) and Steps 30-60 repeated until the focus is in the correct location for HIFU therapy to begin.

The repositioning of the HIFU focus (Step 70 of FIG. 1) can be accomplished either manually or it can be done automatically by using a phased array system.

FIGS. 2 and 3 illustrate the increased scatter of the focus that is received in a diagnostic imaging array operating in either a passive receiving mode or possibly in a pulse/echo mode or any other interleaved passive/active imaging mode. The image that is obtained in the diagnostic imaging array is shown in FIG. 2. The beam pattern like image is superimposed over the traditional ultrasound image in FIG. 2. The position where the beam pattern narrows corresponds to the position of the focus. By moving the focus position, one can see, in real time, the movement of the beam pattern on the image. FIG. 3 illustrates the same effect but with the diagnostic imaging transducer 10 operating in a receive mode only (transmitted power is set to zero).

Thus the present invention provides for interactive, real-time position of the focus as well as inexpensive monitoring of the focus.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. A method for monitoring a position of a focus of a therapeutic transducer for real-time, interactive imaging, the steps comprising:
(a) connecting a therapy and imaging transducer to a patient;
(b) setting an imaging array in one of either a passive mode or in an interleaved passive/imaging mode;
(c) moving focus of said therapy transducer to an approximate desired location on said patient;
(d) turning said transducer on to transmit in a short time, low power, high pressure, continuous wave, to produce a scattering phenomenon at focus of said transducer
(e) said imaging array being operated to image and thereby to detect high pressure therapy focus interacting with said scattering phenomenon in passive mode with receive beamforming;
superimposing detected focus on active imaging mode;
(f) determining if focus is in desired location and if not repositioning said focus of said transducer to said desired location and repeating steps a through f.

2. The method according to claim 1 wherein after said determining step if said focus is not in desired location said transducer is turned off before repositioning said focus of said transducer.

3. The method according to claim 1 wherein said scattering phenomenon is a bubble induced by cavitation at said therapeutic focus.

4. The method according to claim 1 wherein said scattering phenomenon is a local tissue phenomenon.

5. The method according to claim 1 wherein said short time is in the range of micro seconds to tens of seconds.

6. The method according to claim 1 wherein said low power is in the range of 1 milliwatt to 10 watts.

7. The method according to claim 1 wherein said repositioning step is done manually.

8. The method according to claim 1 wherein said repositioning step is done automatically by said imaging array being a phased imaging array.

9. An apparatus for monitoring a position of a focus of a therapeutic transducer for real-time, interactive imaging, the steps comprising:
(a) a therapy and imaging transducer connected to a patient;
(b) an imaging array set to one of either a passive mode or in an interleaved passive/imaging mode;
(c) said therapy transducer having its focus moved to an approximate desired location on said patient;
(d) said transducer set to transmit in a short time, a low power, high pressure, continuous wave, cavitation mode to produce a scattering phenomenon at focus of said transducer
(e) imaging with said array to detect high pressure focus interacting with said scattering phenomenon in passive mode and to superimpose said detected focus on an active imaging mode wherein said detected focus is compared with said active imaging mode to determining if said focus is in desired location and if not said focus of said transducer is repositioned to said desired location and repeating steps d through e.

10. The apparatus according to claim 1 wherein said transducer is turned off before repositioning said focus of said transducer.

11. The apparatus according to claim 9 wherein said scattering phenomenon is a bubble induced by cavitation at said therapeutic focus.

12. The apparatus according to claim 9 wherein said scattering phenomenon is a local tissue phenomenon.

13. The apparatus according to claim 9 wherein said short time is in the range of micro seconds to tens of seconds.

14. The apparatus according to claim 9 wherein said low power is in the range of 1 milliwatt to 10 watts.

15. The apparatus according to claim 9 wherein said repositioning step is done manually.

16. The apparatus according to claim 9 wherein said repositioning step is done automatically by said imaging array being a phased imaging array.
